(19) United States
(12) Patent Application Publication
(10) Pub. No.: US 2010/0260795 A1
(43) Pub. Date: Oct. 14, 2010

(54) Carrier Particle for a Microorganism or Subunit Thereof, Pharmaceutical Composition Comprising such Particles, Method for Preparation of this Composition and Its Use in the Treatment of Animals

(76) Inventors: Erwin Mombarg, Boxmeer (NL); Rogier Biemans, Boxmeer (NL)

Correspondence Address: Intervet/Schering-Plough Animal Health, Patent Dept. K-6-1, 1990, 2000 Galloping Hill Road, Kenilworth, NJ, 07033-0530, US

(21) Appl. No.: 12/744,426

(22) PCT Filed: Nov. 26, 2008
(86) PCT No.: PCT/EP08/66188

§ 371 Date: May 24, 2010

Related U.S. Application Data
(60) Provisional application No. 60/990,819, filed on Nov. 28, 2007.

(30) Foreign Application Priority Data
Nov. 27, 2007 (EP) 07121588.3

(51) Int. Cl. A61K 39/00; A61K 39/12; A61K 39/295; A61P 31/12; A61P 31/04

(52) U.S. Cl. 424/201.1; 424/184.1; 424/204.1

(57) ABSTRACT

The invention pertains to a carrier particle comprising a hydrophilic phase containing a micro-organism and/or subunit thereof, the hydrophilic phase being dispersed in a hydrophobic continuous phase being solid at room temperature, wherein the hydrophobic phase is constituted to undergo a solid-to-liquid conversion at a temperature above room temperature, the conversion comprising a first order transition. The invention also pertains to a pharmaceutical composition comprising said particles, a method for preparation the pharmaceutical composition and the use of this composition in the treatment of an animal.

The present invention pertains to a carrier for a micro-organism and/or subunit thereof, a pharmaceutical composition comprising this carrier, a method for the preparation of the pharmaceutical composition and its use to treat an animal for a disease related to the micro-organism.

It is common practice for protection of animals (including humans) against diseases, in particular transmittable infectious diseases, to administer pharmaceutical compositions, e.g. a vaccine, comprising one or more micro-organisms (i.e. life-forms of micron or submicron size, including bacteria and viruses) and/or sub-units thereof (commonly addressed as “antigens”) that are related to the disease, in order for the animal to be able and effectively develop a defensive response, e.g. when confronted with an infection with the wild-type micro-organism. For this purpose, when a micro-organism is used in the composition, it is customary that the micro-organism is administered in a live but non-virulent form (often referred to as “live attenuated”), or an inactivated form (“killed”). It is also common to use a sub-unit of a micro-organism, i.e. a part of the micro-organism that constitutes an antigenic determinant, capable of inducing an immune response in an animal against the micro-organism itself. A commonly used subunit of bacteria for example is a part of an outer membrane protein. In the cases wherein killed micro-organisms and/or subunits are used, often components are added that stimulate the immune response in the target animal. Such immune response stimulating components are usually referred to by the term “adjuvants”. Different types of adjuvants are known. Many of these are based on mineral oils as immune stimulating agents. Often these oils are formulated with water to form an emulsion. Depending on which phase is the discontinuous or continuous phase, several types of emulsions can be distinguished, e.g. a W/O (water droplets dispersed in oil), O/W (oil droplets dispersed in water) or W/O/W emulsion (water droplets dispersed in oil droplets, again dispersed in water) etc. Adjuvant formulations can be used as the carrier means for carrying the micro-organism and/or subunit thereof in a pharmaceutical composition. It is important that the carrier provides stability during storage, not only with regard to the stability of the micro-organism or sub-unit itself, but also with regard to the physical constitution of the pharmaceutical composition. Moreover, the carrier should allow easy administration, e.g. a low viscosity when injection is the preferred route. Also, it is important that the carrier allows easy formulation of the pharmaceutical composition, e.g. such that a minimal amount of energy is taken up during manufacturing. This can be important since energy uptake usually leads to (at least local) temperature increase which often leads to irreversible change of the micro-organism and sub-unit, which on its turn could lead to loss of capability of inducing an adequate immune response in the treated animal.

From EP 1 179 349 a carrier means for a micro-organism and/or subunit thereof is known that provides stability during storage, allows easy administration by injection (due to its low viscosity) and is easy to formulate. The known carrier means comprises a disperse aqueous phase in a continuous oil phase which is based on a liquid fat or wax. The oil phase comprises silicon dioxide particles which provide for a thixotropic effect in this phase. This way the oil phase, when mechanically unattached, behaves like a highly viscous phase that provides a very good mechanical stability of the emulsion, since the tendency of the oil droplets to coagulate is highly reduced. When shaken, the viscosity drops such that the emulsion allows administration via injection. Also, when the oil phase comprising the silicon dioxide particles is stirred, the viscosity is very low. The thixotropic oil phase thus allows easy formulation without too much energy uptake. The known carrier however has a few important disadvantages. Firstly, silicon dioxide is often associated with toxicity, lesions and abscesses. Therefore, application in a carrier for a pharmaceutical composition is not preferred. Moreover, a depot of solid particles in the recipient's body is not highly appreciated. For human applications the risk is generally regarded too high, for application in breeding stock the depot may lead to a site that is not suitable for human consumption. Secondly, the thixotropic properties of the oil phase provide for an almost immediate regeneration of the high viscosity when the oil phase is no longer mechanically disturbed. This is impractical for administration by injection: a filled syringe has to be shaken well immediately before actual injection, since otherwise the viscosity will be too high for a smooth injection process. This “shake syringe well before injection” is not customary to doctors or veterinarians.

It is an object of the present invention to overcome or at least mitigate the disadvantages of the prior art carrier means, and at the same time preserving as much of the advantages of this carrier means as possible. To this end a carrier particle has been devised, the particle comprising a hydrophilic phase containing a micro-organism and/or subunit thereof, the hydrophilic phase being dispersed in a hydrophobic continuous phase being solid at room temperature, wherein the hydrophobic phase is constituted to undergo a solid-to-liquid conversion at a temperature above room temperature, the conversion comprising a first order transition. In the particle according to the invention, the micro-organism and/or subunit are contained in a hydrophilic phase (hydrophilic in this sense means having an affinity for water such that upon mixing with water a homogenous one phase mixture can be formed), that is dispersed in a continuous hydrophobic phase (hydrophobic being the opposite of hydrophilic), which hydrophobic phase is solid at room temperature, i.e. 25° C. The fact that the hydrophobic phase is solid at room temperature provides for a very good physical stability up to at least room temperature. A very important aspect of the present invention is that the hydrophobic phase is constituted to undergo a solid-to-liquid conversion at a temperature above room temperature, the conversion comprising a first order transition. As is commonly known, a first order transition is a discontinuous transition, i.e. a transition involving a discontinuous change in entropy at the transition point (see P. Ehrenfest, Proc. Acad. Sci. Amsterdam, 36, 153, 1933). This corresponds to a sudden change in properties for the compound undergoing the transition. An example of this type of transition is the melting of ice into water. As ice melts, the “order” in the H₂O molecules changes which provides completely different properties for the substance above and below the transition point. In the present invention, since the solid-to-liquid conversion comprises a first order transition, a very sudden drop in the viscosity above the transition point can be provided for. As long as the temperature is kept above the temperature at which the transition takes place, the viscosity can remain low. This allows easy formulation of the emulsion just above the temperature where the first order transition takes place. In case of a second or higher order transition (or more precisely: a continuous transition) above room temperature, the viscosity of the phase will only drop gradually, which means that a relatively high energy uptake is the usual result (either because the phase is warmed up to relatively high temperatures, or because the viscosity is relatively high). It is noted that the term “solid” in the sense of the present invention means “self-bearing”, i.e. when the “solid” composition is regarded as such, it has sufficient internal strength to macroscopically keep its shape, independent of its physical environment (e.g. the shape of the container it is put in). “Liquid” in the sense of the present patent means that when the “liquid” composition is regarded as such, it has insufficient internal strength to keep its shape, the shape being determined almost immediately by its physical environment (e.g. the shape of the container it is put in). For example, a rubber bouncing ball (although being deformable), a piece of glass (although being amorphous and thus viscous) and some gelled fluids (e.g. concrete, although containing a fluid) can be regarded as being solid, whereas compositions such as molasses, suntan cream and pancake batter (although they can withstand some shear force) can be regarded as being liquid in the sense of this patent application. It is expressly noted that “solid” does not necessarily mean that each compound in the solid composition has to be in a solid constitution. For example, in the case of a solid gel, it may even be that a network of molecules that gives the composition its self-bearing properties, comprises only 10% of the actual mass in the composition whereas the interstices of the network are filled with liquid (thus forming 90% of the composition).

In an embodiment, the first order transition corresponds to a melting process of a crystalline compound, i.e. a compound which upon solidification can form a constitution having a uniform structure in each dimension (but not necessarily the same for each dimension), comprised in the hydrophobic phase. It appears that such a compound is very suitable for application in a carrier particle according to the invention given its inherent stability below the crystallisation temperature and its very swift change into a less ordered constitution above its melting temperature.

In a further embodiment the compound is a metabolisable compound, in particular a fatty acid ester. A metabolisable compound, e.g. a natural wax such as coconut oil (M.p.±26° C.), palm oil (M.p.±35° C.) and mutton tallow (M.p.±42° C.), has the property that it can change by metabolism, in particular the metabolism of the target animal. This mitigates, or even completely solves the problem of a depot which is customary when for example mineral oil is used in the carrier means. A wax in this sense is defined as a substance which at room temperature is solid, upon solidification normally forms crystals, gives off when rubbed by hand and has a melting point below 75° C.

In another embodiment, the hydrophilic phase comprises water and an additional compound. Water is the most commonly used carrier for antigens and indeed, is very acceptable as a constituent in a pharmaceutical composition. In this embodiment, a second compound is present next to the water. It has been seen that this can lead to an improvement of the stability of the micro-organism or subunit as such. In a further embodiment, the additional compound is a poly-alcohol, preferably glycerol.

In another embodiment the hydrophobic phase contains a second micro-organism and/or subunit thereof, which second micro-organism and/or subunit thereof maybe the same as or different from the first micro-organism an/or subunit thereof. This embodiment allows a higher load of the particle with antigens, since a greater part of the particle can be used for actually carrying micro-organisms and/or subunits thereof. Moreover, micro-organisms or subunits that react when in contact, which for example leads to a less adequate immune response when administered to an animal, can be kept apart by putting them in two separate phases which are superior in withstanding exchange of content which is the case with the carrier particles of the present invention. Also, by putting antigens in the two separate phases, a timing difference in the release of the antigens can be provided for. This allows for example the formulation of a one-shot pharmaceutical preparation that upon application mimics the effect of a primer/booster administration.

The present invention also pertains to a pharmaceutical composition for treating an animal, comprising carrier particles according to the invention. Treating an animal in this case also includes the treatment of unborn animals such as chicken embryo's. The particles can be used as such, for example in the form of molten droplets administered to the animal, but in an embodiment the pharmaceutical composition comprises a continuous hydrophilic phase wherein carrier particles as described above are dispersed. The viscosity of this composition is for a substantial part determined by the viscosity of the second hydrophilic phase, which for example can be water, optionally combined with one or more other fluids, and optionally comprising dissolved and/or dispersed material giving the hydrophilic phase an osmolarity that is pharmaceutically acceptable, in particular not causing macroscopically noticeable tissue damage in the animal to be treated. The fact that the viscosity of this composition is for a substantial part determined by the viscosity of the second hydrophilic phase, is an important improvement over the prior art pharmaceutical composition wherein the viscosity is mainly determined by the mechanical disturbance of the system. It is noted that “treating” in the sense of the present invention comprises actions taken aimed at preventing, diagnosing, curing and providing relief for a disease or symptoms related to this disease. Apart from the components as described here-above, the pharmaceutical composition (in one or more of the two or three separate phases) can comprise all sorts of aids such as emulsifiers, stabilisers, antioxidants, adjuvants (such as aluminium salts, immunostimulating complexes, saponins, derivatives of lipopolysaccharides, mycobacteria etc), traceable compounds for diagnostic purposes, salts for buffering or other purposes, etc.

It is noted that from EP 1 097 721 a pharmaceutical composition having good storage stability is known, which composition comprises as a carrier W/O/W emulsion, wherein the hydrophobic phase (“O”) is solid at a temperature below room temperature. At room temperature however the hydrophobic phase of this known composition is liquid (in fact, in all exemplified compositions, the hydrophobic phase is liquid even at a temperature as low as 0° C.). Next to this, above room temperature the hydrophobic phase does not undergo a first order transition, necessitating (in order to allowing formulation of the W/O emulsion without to much mechanical effort) heating up the hydrophobic phase to relatively high temperatures ranging from 53° C. to 125° C. above the melting temperature of the main constituent of the hydrophobic phase. This composition is therefore removed further away from the present invention than the composition as known from EP 1 179 349.

In an embodiment, the hydrophobic phase is constituted such that the first order transition takes place at a predetermined temperature with respect to a body temperature of the animal. This embodiment has the very important advantage that the release of the micro-organism and/or subunit can be highly controlled. Applicant namely recognised that the release depends on the moment or speed at which the first order transition takes place in the target animal or even whether or not the first order transition takes place at all in the target animal. On its turn, this will substantially depend on the temperature of the body at the site where the carrier particles will be after administration (for example in a muscle when the pharmaceutical composition is administered intramuscular, in the gastro-intestinal tract when administered orally, in the blood when administered intravascular). These insights were combined by applicant and served as a basis to devise this embodiment wherein the temperature at which first order transition takes place is not an outcome with respect to a body temperature of the target animal, but is actually determined by the wish of being a certain number of degrees away, or the same, as a body temperature (i.e. a temperature which occurs at a site where the carrier particles are localised after administration of the pharmaceutical composition) of that animal.

In an embodiment the first order transition takes place at a temperature below a body temperature of the animal. In this embodiment, the release of the antigens can be relatively quick since the hydrophobic phase will become liquid almost immediately, or at least very soon, after administration to the animal given the fact that the composition will readily be warmed up to reach the body temperature. After the solid-to-liquid transfer, if the resulting W/O type emulsion is unstable, the release of antigens will be almost immediate. The more stable the emulsion is, the longer it will take for all the antigenic material to be released to the animal's body. In some cases, when the emulsion is superior in stability, the release can take as long as for example three months in total.

In another embodiment the first order transition takes place at a body temperature of the animal. “At a body temperature” in this sense means to differ not more than 1 degree from the temperature of the animal body at the site where the carrier particles are localised after administration of the pharmaceutical composition. This way, a slow, long lasting release can be provided for (the duration of which i.a. depends on the stability of the emulsion that is obtained after the first order transition has taken place), for example creating an immune response in the animal that is comparable with the response obtainable with a so called prime-boost vaccination.

In yet another embodiment the first order transition takes place at a temperature above a body temperature of the animal. This way, a very slow or even deferred release can be provided for. It is for example possible to choose a temperature at which the first order transition takes place which is only reached in the animal's body when the animal has a fever. Before the actual transition takes place, release is possible for example through diffusion of the antigens through the solid hydrophobic phase. Such diffusion, depending inter alia on the type of antigen and the type of hydrophobic phase, can be completely barred or very slow, with intermediate speed or relatively fast. An alternative route for release in this embodiment can be provided for when the hydrophobic phase is metabolised by the animal. This way, pathways for release can be created in the carrier particles. These embodiments can be used for example when the release should take more than 3 months in total, or should be deferred, e.g. until an animal develops a fever or undergoes another process that induces (local) temperature raise.

The invention also pertains to a method for preparation of a pharmaceutical composition comprising admixing a micro-organism and/or subunit thereof in a first hydrophilic phase, emulsifying the resulting mixture in a hydrophobic phase that is able to undergo a solid-to-liquid conversion above room temperature, at a temperature above the temperature at which the solid-to-liquid conversion takes place, resulting in a single emulsion (i.e. an emulsion wherein one phase is dispersed in another phase) of hydrophilic phase droplets in the continues hydrophobic phase, mixing the resulting single emulsion with a second hydrophilic phase at a temperature above the temperature at which the solid-to-liquid conversion takes place, resulting in a double emulsion (i.e. an emulsion wherein one phase is dispersed in another phase which on its turn is dispersed in yet another phase) in which the second hydrophilic phase becomes the continuous phase of the pharmaceutical composition, and cooling the double emulsion to a temperature below the temperature at which the solid-to-liquid conversion takes place.

In an embodiment of this preparation method the second hydrophilic phase comprises a non-aqueous compound, preferably a polyalcohol, more preferably glycerol. Applicant has found that this way a double emulsion can be obtained in which the disperse hydrophobic droplets have a small size (typically below 50 μm) and a very narrow particle distribution, e.g. 20 μm±10 μm (d95, volume averaged).

In an embodiment the cooling takes place by mixing the double emulsion with a water containing fluid that has a temperature below the temperature at which the solid-to-liquid conversion takes place. This is a very convenient way to obtain the pharmaceutical composition.

The invention also pertains to the use of a pharmaceutical composition as described here-above to treat an animal for a disease related to the micro-organism.

It is noted that the present inventions are not restricted to a particular type of micro-organism or subunit thereof. In principle, the present invention can be used in combination with any micro-organism and/or subunit thereof. The invention will now be further explained by using the examples and figures as referred to here-beneath.

Example 1 Method of preparation carrier particles and a pharmaceutical composition according to the invention.

Example 2 use of carrier particles containing Actinobacillus pleuropneumoniae (APP) antigens.

Example 3 use of carrier particles containing Porcine circo virus antigens.

Example 4 use of carrier particles containing avian viral and bacterial antigens.

Example 5 Characterisation of the hydrophobic phase of the carrier particles.

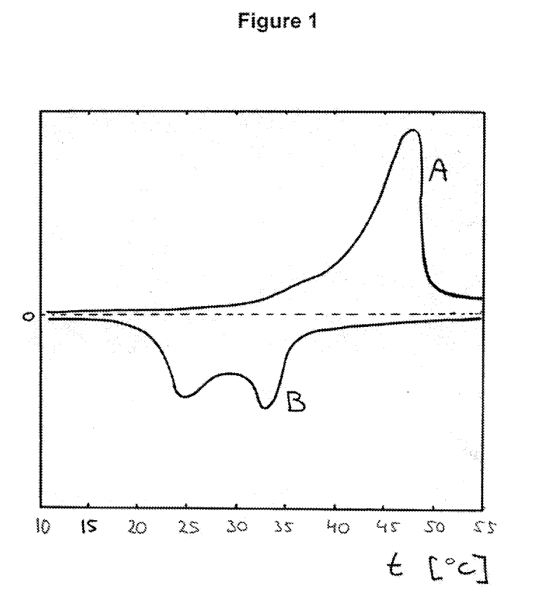

FIG. 1 DSC image of Witepsol E85

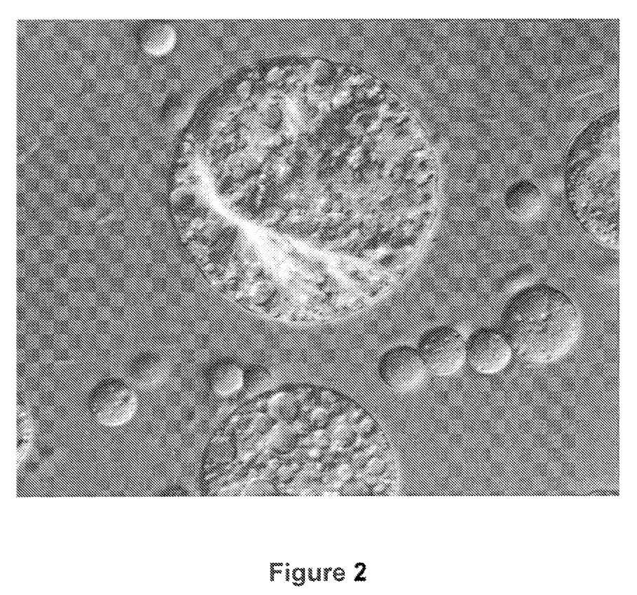

FIG. 2 Picture of a carrier particles according to the invention.

### EXAMPLE 1

In this example a method for obtaining carrier particles and a pharmaceutical composition according the invention is described. At first a buffer solution is made by adding 0.44 g of the Inutec SP1 surfactant (Orafti, Belgium) to 10.56 g of a buffer solution. The resulting mixture (“Mixture 1”) is stirred using a magnetic stirring bar for 1 hour. Then, Mixture 1 is autoclaved for 1 hour at 121° C. without stirring (Varioklav, H+P Labortechnik, Germany). After that, the autoclave is turned off, the door is opened and Mixture 1 is left to cool down to 48° C. while stirring. This takes approximately 1 to 2 hours.

During the cooling down process of Mixture 1, a next mixture (“Mixture 2”) is being made by adding 7.83 g Witepsol E85 (Sasol, Germany) to 0.16 g Arlacel P135 (Uniqema, The Netherlands), heating the mixture to 50° C. and thereafter sterilize the mixture by 0.22 μm filtration with a filter suitable for oily solutions (such filters are available for example from Pall, Sigma Aldrich and Millipore). The resulting Mixture 2 is stored at 48° C. till use.

A sterile mixture of antigens is prepared by bringing antigens in a sterilized buffer. The amount of antigens in the buffer depends on the desired amount of antigen units in the final vaccine. This antigen mixture (“Mixture 3”) is stored at room temperature till use.

Mixture 3 is heated up quickly to 48° C. by using a water batch with a temperature of about 60° C. The heating process should preferably take less than 5 minutes. In the mean time, 7.26 g of Mixture 2 is homogenized at 48° C. using an ultra turrax (T25B, IKA Labortechnik, Germany) at 24.000 rpm. A next mixture is prepared by slowly adding of 4.84 g of Mixture 3 to 7.26 g of Mixture 2, which should take approximately 2 minutes. The resulting mixture is homogenized at 24.000 rpm till the quality of the mixture (“Mixture 4”) is acceptable. The quality is of the warm product checked by using a standard light microscope (Olympus BX50), using object glasses (also called sample plates) that are preheated to a temperature slightly above 48° C. The quality of the mixture is acceptable when 95% of the antigen phase droplets are smaller than 5 μm. Mixture 4 is stored at 48° C. for 10 minutes.

Then, Mixture 1 is homogenized at 48° C. using an ultra turrax (T25B) at 24.000 rpm. To this Mixture 1, 11.00 g of Mixture 4 is slowly added during a period of 3 minutes. The homogenization is stopped as soon as the specifications are met. The specifications are: 99% of the particles are smaller than 80 μm (checked with the same light microscope as described before). The obtained product (“Mixture 5”) is stored at 48° C. for a short period of time (typically less than 5 minutes). This product is a W/O/W emulsion of which the oil (hydrophobic Witepsol) is a liquid. The density of the oil droplets in Mixture 5 is relatively high. In order to prevent agglomeration of the oil droplets upon cooling of Mixture 5, the mixture is cooled in a buffered and continuously stirred solution, such that cooling of the droplets to become solid spheres is accompanied by dilution of the droplets. This buffered solution is made by mixing 0.54 g of a 10% solution of formaldehyde in the same buffer as used to prepare Mixture 1, 11.44 g of the adjuvant Microsol Diluvac Forte (“MDF” adjuvant, as used in the products Myco Silencer Once and End-FLUence 2, Intervet USA) and 66.65 g of the same buffer as used to constitute Mixture 1, and cool this mixture to 5° C. This mixture (“Mixture 6”) is stirred at 100 rpm (Euro-STP CV agitator, IKA Labortechnik, Germany). To this Mixture 6, 20.00 g of Mixture 5 (that was being stored at 48° C.) is added. The resulting pharmaceutical composition is kept below 8° C., filled in vials and stored at 2-8° C. till vaccination. Note that instead of the MDF adjuvant it can be decided to leave out the adjuvant and replace it by e.g. buffered sterile water, or to use any other adjuvant, for example one or more of the adjuvants as described in EP 382271 or EP 1613346.

### EXAMPLE 2

For this experiment the antigens as known from the commercially available vaccine Porcillis APP (available from Intervet, Boxmeer, The Netherlands) were used, i.e. ApxI, ApxII, ApxIII and OMP. These antigens were brought in a sterile Tris-HCl buffer (40 mM trishydroxymethylaminomethane, brought to pH 7.5 with HCl) to obtain Mixture 3 as outlined in Example 1 (note: the same buffer is used to obtain Mixture 1). This Mixture 3 contained 250 Units of each antigen per ml. This ultimately resulted in a formulation containing 10 Units of APP antigens per ml, as compared to 25 Units per ml of the commercially available product.

The animals used for the test were 6 weeks old piglets. Six piglets received 1 ml of the formulation obtained as described here-above, by intramuscular injection in the neck. The booster vaccination took place after 4 weeks. Six piglets were given the commercially available Porcillis APP vaccine, also at 6 and 10 weeks of age, by intramuscular injection of 2 ml of the vaccine in the neck. A control group of six piglets were given 2 ml of phosphate buffered saline at 6 and 10 weeks of age, by intramuscular injection in the neck. The animals were tested for local reactions, rectal temperature, clinical signs and antibody titers against Actinobacillus pleuropneumoniae.

Some of the piglets that were given the APP vaccine showed mild clinical signs such as shivering, vomiting and/or increased respiration and occasionally a local reaction after the first and second vaccination. The animals that were given the formulation comprising the carrier particles according to the invention showed no clinical signs or local reactions at all, as was the case with the control animals. Rectal temperature, when compared to the control animals rose slightly for the formulation comprising the carrier particles. The maximum difference was 0.7° C. after the first vaccination and 1.1° C. after the second vaccination. This is within acceptable levels and equal to or even less then the rectal temperature increase when Porcillis APP is being administered. The antibody titers obtained are shown in table 1. These titers are normalized with respect to the titers as obtained with the commercially available product Porcillis APP

TABLE 1
Antibody titers for the various antigens, normalised with respect
to the titers as obtained with Porcillis APP.

| Product | Apx I | Apx II | Apx III | OMP |
|---|---|---|---|---|
| Porcillis APP | 1.0 | 1.0 | 1.0 | 1.0 |
| New formulation | 0.5 | 2.1 | 1.4 | 1.4 |
| Control | 0.0 | 0.0 | 0.0 | 0.0 |

As one can see, despite the fact that with administration of 1 ml of the new formulation, only about 20% of the antigens is injected when compared with 2 ml of the Porcillis APP vaccine, titers are comparable or even slightly improved.

### EXAMPLE 3

For this experiment Porcine circo virus Type 2 antigens were used. These antigens are the ORF 2 encoded protein of PCV 2, expressed in a baculo virus expression system as commonly known in the art, e.g as described in WO 2007/028823. Whole cell lysate is used, buffered in SF-900 II SFM medium (available from Invitrogen, USA) to obtain Mixture 3 as outlined in Example 1. This mixture contains 100.000 (Elisa) Units of antigen/ml (2,5E03 of these Units are equal to 20 μg ORF2 encoded protein). To obtain the ultimate formulation, the method according to Example 1 was followed, with the following alterations: to obtain Mixture 1, 0.20 g of Inutec was added to 9.80 g of SF-900 II SFM (as a buffer); to obtain Mixture 2, 9.80 g of Witepsol H185 and 0.20 g of Arlacel P135 were used; to obtain Mixture 4, 5.00 grams of Mixture 3 and 5.00 grams of Mixture 2 are used; to obtain Mixture 5, 10.00 grams of Mixture 4 is added to Mixture 1; the buffer solution to obtain Mixture 6 was made by mixing 0.68 grams of PCV antigen concentrate (containing 221528 U/g) with 16.72 g MDF and 35.32 g SF-900 II SFM; to this buffer solution 6.00 grams of Mixture 5 is added to obtain Mixture 6. This ultimately resulted in a formulation containing, per ml product, 2500 Units of PCV antigens in the hydrophobic phase and 2500 Units of PCV antigens in the continuous hydrophilic phase of the W/O/W double emulsion.

The animals used for the test were 2 weeks old piglets. Ten piglets received 2 ml of the formulation as obtained according to example 1, by intramuscular injection in the neck. Ten piglets were given a PCV vaccine made according to WO 2007/028823 containing 5000 Units per dose by intramuscular injection of 2 ml of the vaccine in the neck. Those piglets received the same vaccination as a booster vaccination two weeks after the first vaccination. A control group of 10 piglets were given 2 ml of SF-900 II SFM by intramuscular injection in the neck. The animals were tested for local reactions and antibody titers against Porcine circo virus until 8 weeks after the booster vaccination with the known vaccine.

With some of the piglets (3) that received the new formulation, remains of this formulation some were visible at the end of the experiment. The development of the antibody titers obtained with the new vaccination, despite the fact that no booster vaccination was given, was more or less the same as with the known vaccine, albeit that the obtained titer levels were restricted to a maximum of about 80% of the level (on a logarithmic scale) as obtainable with the known vaccine. Still, these titers are sufficient to provide a significant level of protection for the piglets.

### EXAMPLE 4

For this experiment a combination of viral and bacterial avian antigens is used. These antigens are the same antigens as present in the vaccines Nobilis IB multi+ND+EDS (first viral vaccine), Nobilis RT Inac (second viral vaccine) and Nobilis Salenvac T (bacterial vaccine), all available from Intervet, Boxmeer, The Netherlands. The ultimate formulation, per ml of product contains the same amount of antigens as is the case with the commercially available vaccines. In order to obtain this formulation, the antigens of the first two (viral) vaccines were suspended in 8.56 gram sterile water to obtain Mixture 3. Mixture 1 was made by using 0.96 grams Inutec SP1 and 47.04 grams of sterile water (not buffered). Mixture 2 was obtained by using 24.50 grams of Witepsol E85 and 0.50 grams of Arlacel P135. Mixture 4 was obtained by using 16.5 grams of mixture 3 and 16.50 grams of Mixture 2. Mixture 5 was obtained by using 33.0 grams of Mixtures 1 and 3 respectively. The buffer solution to obtain Mixture 6 was made by mixing 0.26 grams of Trometamol (available from Merck, Germany), 0.25 grams of maleic acid (available from Sigma Aldrich), 0.90 grams of sodium chloride (available from Merck), 79 ml of sterile water, 27.50 grams of aluhydroxide gel (available from Brenntag Nordic, Sweden) and killed salmonella bacteria as present in Nobilis Salenvac T. Mixture 6 was ultimately obtained by using 22.00 grams of Mixture 5 in addition to this solution.

This formulation was used to vaccinate 4 weeks old chickens. A first group of 10 chickens were vaccinated with 0.5 ml of the new formulation into the left breast muscle. A second group of 10 chickens received the commercially available vaccines Nobilis IB multi+ND+EDS and Nobilis RT Inac. A third group of ten chickens received the commercially available product Nobilis Salenvac T. The birds were bled at 4 and 6 weeks post vaccination and the collected sera were tested for antibodies against the antigens. When comparing the new combined formulation with the existing products, it appeared that good antibody titers could be obtained against the IB, RT and Salmonella antigens. The antibody titers against the EDS and ND antigens however were low when compared to the titers obtainable with the existing products.

### EXAMPLE 5

A material suitable for use in the hydrophobic phase of a carrier particle according to the invention must undergo a solid-to-liquid conversion at a temperature above room temperature, the conversion comprising a first order transition. Such a material can be found, for example by screening materials allegedly having a melting point or range above room temperature in a differential scanning calorimeter (DSC), for example the Perkin Elmer DSC 7, in order to find out whether the transition indeed comprises a first order transition. A method suitable to detect such a transition is to subject the sample to a first heating cycle to rule out any thermal history effects, for example by heating the sample to a temperature of more than 10° C. above it's melting temperature at a speed of 5° C./min and then cool the sample to 10° C. at the same speed. Then, a second heating cycle is used to establish whether or not the material undergoes a first order transition above room temperature. This heating cycle may comprise heating the sample at a speed of 5° C. to a temperature of 10° above it's melting temperature and then cool the sample to 10° C. at the same speed. This method has been used to select materials suitable for constituting carrier particles according to the invention. In FIG. 1, a DSC diagram of Witepsol E85 is shown, the diagram being obtained by using the same method

FIG. 1

FIG. 1 shows a DSC diagram of Witepsol E85, measured in accordance with example 5 (X-axis gives the temperature in ° C.; Y-axis gives the heat flow H in arbitrary units). Witepsol E85 is a mixture of compounds which gives rise to various transitions when this material is warmed up from 10° C. to about 60° C. Given the asymmetrical shape of the melt peak A, two first order transitions take place when melting the Witepsol compound. The melting points are approximately 40 and 47° C. respectively. Moreover, it is believed that at approximately 35° C. a higher order transition takes place given the slight “bulge” at this temperature and a rise in the baseline when going from the left to the right of the peak A). In total, these transitions show as one broad peak that starts at around 30° C. and ends at around 50° C. When cooling the molten material, it starts to crystallize at a temperature just above 35° C. The fact that two distinct crystallization peaks B are determined, one at around 33° C. and the other at around 24° C., corresponds with the appearance of two first order transitions as seen when melting Witepsol E85.

Although in the present examples Witepsol E 85 and H185 are used to constitute the carrier particles according to the invention, it may be clear that other materials can be used, as long as they are hydrophobic and undergo a solid-to-liquid conversion at a temperature above room temperature, wherein the conversion comprises a first order transition. Such materials are for example Witepsol H5 (melting range approximately 34-36° C.), or branched alcohols such as ISOFOL 28 (melting range 32-39° C.) and ISOFOL 32 (melting range 44-48° C.). The latter two materials are also available from Sasol (Germany). Other suitable materials are for example hydrogenated oils such as hydrogenated Castor oil, cetyl palmitate, the higher melting oleyl alcohols (up to 35° C.), numerous triglycerides and the hardened fats as available from Cognis (Monheim Germany) under the tradename Novata, part of their Pharmaline or Edenor L2 SM GS, a vegetable based stearic-/palmitic acid also available from Cognis. Other materials are for example oils that are liquid at room temperature, but which oils comprise a crystalline gelling agent, which agent melts (undergoing a first order transition) above room temperature. This way, the main components of the hydrophobic material is a liquid, but is caged in the interstices of the network of gelled molecules of the gelling agent. Such a gelled oil is in fact a (semi-)solid carrier particle which becomes fluid upon the melting of the gelling agent. Preferably, the material or materials used for constituting the hydrophobic phase are pharmaceutically acceptable, i.e. they do not evoke significant physical problems when administered in a pharmaceutical composition. More preferably, the materials are recognised pharmaceutical excipients. In practice, in particular for use in mammals, typical temperatures for the first order transition are below 60° C.

FIG. 2

FIG. 2 is a microscopic picture of carrier particles according to the invention. The emulsion of particles as obtained under example 2 is stirred for 15 minutes at 1000 rpm at a temperature of 39° C. Then a sample is taken and put on a transparent sample plate suitable for a light microscope. Immersion oil is added and the sample is covered by a second transparent sample plate. Then the image as depicted in FIG. 1 can be obtained using a regular light source in a transmission mode of a regular light microscope. The smallest particles in FIG. 2 are around 5 μm in diameter. The large particle in the center has a diameter of around 50 μm. These particles consist of a hydrophobic continuous phase and have dispersed therein aqueous droplets, typically of a size between 0.5 and 5 μm, comprising the APP antigens.

1. A carrier particle comprising a hydrophilic phase containing a micro-organism and/or subunit thereof, the hydrophilic phase being dispersed in a hydrophobic continuous phase being solid at 25 degrees C., wherein the hydrophobic phase is constituted to undergo a solid-to-liquid conversion at a temperature above 25 degrees C., the conversion comprising a first order transition.

2. The carrier particle according to claim 1, wherein the first order transition of the hydrophobic phase corresponds to a melting process of a crystalline compound comprised in the hydrophobic phase.

3. The carrier particle according to claim 2, wherein the hydrophobic phase is a metabolisable fatty acid ester.

4. The carrier particle according to claim 1, wherein the hydrophilic phase comprises water and an additional compound.

5. The carrier particle according to claim 4, wherein the additional compound is a poly-alcohol.

6. The carrier particle according to claim 1, wherein the hydrophobic phase contains a second micro-organism and/or subunit thereof.

7. A pharmaceutical composition for treating an animal, comprising carrier particles according to claim 1.

8. The pharmaceutical composition according to claim 7, comprising a continuous hydrophilic phase in which carrier particles are dispersed.

9. The pharmaceutical composition according to claim 7, wherein the hydrophobic phase is constituted such that the first order transition takes place at a predetermined temperature with respect to the body temperature of the animal.

10. The pharmaceutical composition according to claim 9, wherein the first order transition takes place at a temperature below the body temperature of the animal.

11. The pharmaceutical composition according to claim 9, wherein the first order transition takes place at the body temperature of the animal.

12. The pharmaceutical composition according to claim 9, wherein the first order transition takes place at a temperature above the body temperature of the animal.

13. A method for preparation of a continuous phase pharmaceutical composition comprising:
admixing a micro-organism and/or subunit thereof with a hydrophilic medium in a first hydrophilic phase,
emulsifying the resulting mixture in a hydrophobic phase that is able to undergo a solid-to-liquid conversion above room temperature, at a temperature above the temperature at which the solid-to-liquid conversion takes place, resulting in a single emulsion of hydrophilic phase droplets in a continuous hydrophobic phase,
mixing the resulting emulsion with a second hydrophilic phase at a temperature above the temperature at which the solid-to-liquid conversion takes place, resulting in a double emulsion in which the second hydrophilic phase becomes the continuous phase of the pharmaceutical composition,
cooling the double emulsion to a temperature below the temperature at which the solid-to-liquid conversion takes place.

14. The method according to claim 13, wherein the second hydrophilic phase comprises a non-aqueous compound.

15. The method according to claim 14, wherein the cooling of the double emulsion takes place by mixing the double emulsion with a water containing fluid that has a temperature below the temperature at which the solid-to-liquid conversion takes place.

16. A method for inducing an immune response in an animal comprising administering an immunogenically effective amount of the pharmaceutical composition according to claim 7.

17. The carrier according to claim 4, wherein the additional compound is glycerol.

18. The method according to claim 14, wherein the non-aqueous compound is a polyalcohol.

19. The method according to claim 18, wherein the polyalcohol is glycerol.
